(19) United States
(12) Patent Application Publication
(10) Pub. No.: US 2008/0249387 A1
(43) Pub. Date: Oct. 9, 2008

(54) Enhanced monitor compliance

(76) Inventor: Josh N. Hogan, Los Altos, CA (US)

Correspondence Address: Josh Hogan, 620 Kingswood Way, Los Altos, CA, 94022, US

(21) Appl. No.: 12/080,318

(22) Filed: Apr. 2, 2008

Related U.S. Application Data
(60) Provisional application No. 60/921,588, filed on Apr. 3, 2007.

(51) Int. Cl. A61B 5/00

(52) U.S. Cl. 600/365

(57) ABSTRACT

A monitoring system that enables enhanced compliance with a bio-sign monitor includes a method of transferring data to a consumer device, such as a cell phone or MP3 player and incorporating information related to the bio-sign data into the normal communication channel used by the consumer device. The combination of monitor and consumer device enables bio-sign related information to be made available in convenient and discreet manner that enhances the likelihood of compliance with the counter measures indicated by the bio-sign monitor.

### CROSS REFERENCES TO RELATED APPLICATIONS

This patent application, docket number JH080327US claims priority from provisional patent application 60/921,588 (docket number JH051104PR), filed on 3, Apr. 2007, the contents of which are incorporated for all purposes herein by reference as if fully set forth herein.

This patent application, relates to U.S. utility application Ser. No. 11/048,694 filed on Jan. 31, 2005 titled “Frequency Resolved Imaging System” which is a continuation in part of U.S. utility application Ser. No. 11/025,698 filed on Dec. 29, 2004 titled “Multiple reference non-invasive analysis system”, the contents of which are incorporated by reference as if fully set forth herein. This application also relates to utility patent application Ser. No. 10/870,121 filed on Jun. 17, 2004 titled “A Non-invasive Analysis System”, the contents of which are incorporated by reference as if fully set forth herein. This application also relates to utility patent application Ser. No. 11/254,965 filed on Oct. 19, 2005, issued as U.S. Pat. No. 7,248,907, titled, “Correlation of Concurrent Non-invasively Acquired Signals”, the contents of which are incorporated by reference as if fully set forth herein.

### FIELD OF THE INVENTION

The invention relates to continuous monitoring of one or more bio-signs, such as glucose concentration level, that is desirable for on-going health care and also relates to wearable consumer devices, such as a cell phone or MP3 players and their interaction in a manner that enhances the likelihood of complying with the behavior indicated by the bio-sign monitor.

### BACKGROUND OF THE INVENTION

Bio-sign monitors, such as glucose concentration monitors, are used by individuals with a problematic medical condition, such as diabetes, to monitor the problematic condition and determine a corrective course of action to control the problematic condition and thereby reduce the effect of negative aspects of the problematic condition. Bio-sign monitors are also more generally used to provide information useful in promoting health and fitness.

In the case of a glucose concentration monitor, corrective courses of action include taking insulin if the glucose concentration is abnormally high and ingesting sugar or glucose if the glucose concentration is low. Glucose monitors that continuously (or very frequently) make glucose concentration measurements, herein referred to as continuous glucose monitors, provide the opportunity to accurately control glucose concentration levels.

Ideally a continuous glucose level monitor would be used as the feed-back system for an insulin delivery system, such as an implanted or indwelling insulin delivery system. Such a combination of monitor and insulin delivery system, often referred to as an “artificial pancreas” is not available to many diabetics and therefore such diabetics must frequently read (or otherwise interact with) a glucose level monitor to determine the appropriate course of action.

This requirement to actively initiate an interaction with the monitor reduces the value of the continuous monitoring and may lead to sub-optimal control of the diabetic condition, despite the fact that sufficient information is available. This sub-optimal aspect can be exacerbated by many factors including: in-attention of the diabetic due to involvement in other activities, or reluctance to obviously engage in a medical related procedure for social reasons, such as peer pressure or inappropriateness of public medical treatment.

Individuals in business meetings or individuals, (particularly adolescents subject to social pressures), may actively or passively decide to forgo checking the monitor for such reasons and thereby risk not receiving critical information in a timely manner. Such non-compliance in appropriately interacting with the glucose monitor and following the appropriate course of action can lead to significant sub-optimal control of glucose concentration levels on an on-going basis.

Sub-optimal control of glucose concentration levels can allow large excursions to occur in the glucose concentration level. Such excursions can cause cumulative, irreversible damage to organs. Such damage can cause severe health consequences, including blindness, kidney failure, heart disease or stroke and can have potentially fatal health consequences.

In cases of extreme glucose level excursions the diabetic may be rendered incapable of appropriately interacting with the glucose monitor or following the appropriate course of action. In such cases the information to address the medical condition is available, but as a practical matter is not usefully available.

Despite the dire medical consequences of poor compliance with bio-sign monitors, various factors operate to reduce compliance. Such factors include: practical everyday activities that make it inconvenient to comply; powerful pressures of social circumstances cause a reluctance to obviously engage in a medical related procedure; and the fact that there may be no immediate conspicuous consequences.

A realistic repercussion of the circumstances described above is that, although the information is available to correctly control a medical condition, such as diabetes, poor compliance routinely occurs. Such poor compliance can inexorably lead to dire medical consequences. There is therefore an unmet need for a convenient, unobtrusive and discreet method of enabling or enhancing compliance with bio-sign monitors.

### SUMMARY OF THE INVENTION

The invention provides a method, apparatus and system for enabling or enhancing compliance with a bio-sign monitor. In particular the invention relates to continuous monitoring of one or more bio-signs, such as glucose concentration level, that is desirable for on-going health care and to consumer devices, such as a cell phone or MP3 player. The invention couples the monitor and the consumer device in a manner that enhances the likelihood of counter measures indicated by the bio-sign monitor being complied with.

### BRIEF DESCRIPTION OF THE DRAWINGS

FIG. 1 is an illustration of a system that enables enhanced compliance with a bio-sign monitor according to the invention.

FIG. 2 is an illustration of an alternative embodiment of the invention.

### DETAILED DESCRIPTION OF THE INVENTION

The present invention is a method, apparatus and system for enabling or enhancing compliance with a bio-sign monitor by making the data generated by the monitor available in a convenient, unobtrusive and discreet manner to one or more individuals or entities that are in a position to follow a course of action that is appropriate to the data generated by the monitor.

In the particular case of the bio-sign being monitored is a glucose concentration level, continuous or very frequent monitoring is highly desirable because glucose levels can change significantly in a matter of minutes. Monitors typically have a visual display to indicate the glucose level or even an audible alarm to indicate an extreme glucose level excursion. Frequently normal daily activities or social pressures inhibit diabetics from interacting with the monitor and to even ignore alarms because of background noise, alarm being muted, or social pressures.

It is, however, appropriate and socially acceptable to interact with many consumer devices in various situations. For example: adolescents can and do continuously interact with MP3 players, iPods or similar audio or audio/visual consumer devices during any occasion or activity; businessmen can and do constantly interact with PDAs (personal digital assistants) or lap-top computers during business meetings; cell phone text messaging, with muted ringer and in vibration mode, is socially acceptable in most situations, even in theaters or during lectures.

In the present invention, the bio-sign monitor determines the proximity, availability and suitability of one or more consumer devices and transfers the bio-sign data to one or more consumer devices associated with the individual (for example a diabetic) being monitored by the bio-sign monitor. The consumer device makes the data available to the diabetic in a convenient and discreet or unobtrusive manner.

The bio-sign monitor continues to monitor for the anticipated changes in the bio-signs being monitored due to appropriately dealing with the original bio-sign data. If the anticipated changes substantially occur then the monitor continues in its normal mode. Failure to monitor the anticipated changes causes the monitor to escalate the priority and obviousness with which bio-sign data is made available to the diabetic.

For example, a bio-sign monitor with a relatively short range wireless or RF channel, such as Bluetooth, would establish a communication link with one or more consumer devices, such as an iPod, a lap-top and a cell phone. Establishing a valid communication wireless channel would establish the proximity and availability of a particular device. Standard authentication and encryption techniques could authenticate the o consumer device and ensure appropriate protection of the data.

In the case of an iPod, the data could be inserted as an audio message thereby making the bio-sign data or information conveniently and unobtrusively available to the diabetic. Initially the audio could be inserted (softly) at suitable breaks in the audio being listened to, such as between tracks or songs, extending the duration of the break if necessary. If the monitor does not detect the anticipated changes (which should be an improvement in the bio-sign) in a timely manner. The audio message could be made more loud and made to impact the iPod experience.

Optional parental control techniques could be employed to add tamper resistance to insertion of the audio message and its escalating impact on enjoying the iPod experience. This provides a method for diabetics to discreetly get access to glucose concentration data and guidance in appropriate counter measures, such as administering a particular insulin or glucose dose. It also provides a mechanism for ensuring that if the diabetic does not take appropriate counter measures there is prompt disagreeable feedback to encourage compliance.

In the case of a lap-top computer, a pop up icon could be used to alert the diabetic to the fact that new or urgent data was available. Reacting to the icon could be readily communicated to the monitor automatically, (verifying that it had contact). Failure by the monitor to detect the anticipated changes could cause larger, or more frequent, or annoying or adding audio alarms as a way of escalating the impact of the communication.

While parental control techniques could be employed in the case of a laptop, more likely the owner of the lap-top would decide on the appropriate manner in which an escalating alarm level should occur. In both the lap-top and iPod cases protocols could readily be developed and integrated into these devices to enhance feedback to the monitor (for example, verify that the device is in active use) and to facilitate appropriate and escalating priority.

In the cell-phone case, an appropriate communication channel is the text messaging channel. This can be a discreet information channel by having the audio aspects of ring tone and beeper or buzzer muted. The vibration mode is still available to indicate that a message has been received. Personalized aspects of cell phones, such as ring tones specific to individual callers can be used to identify and give priority to the monitor communication.

Escalating aspects could include, but are not limited to; inserting an audio reminder with any activity of the cell phone; generating a repeated and more loud beeping indicator that there is an urgent message; generating visual indicators such as a flashing display or a flashing icon on the display; or having such a flashing icon increase in size; having the keyboard illuminators flash in a very obvious and particular manner.

The transferred data can include specific recommendations for counter measures that include taking a dose of insulin or glucose. Glucose can be readily taken orally and digested. Heretofore insulin has been typically injected sub-cutaneously, however, more discreet delivery mechanisms, such as, pulmonary delivery, buccal delivery and orally delivery of insulin facilitate more discreet counter measures.

In the particular case of orally delivery of insulin encapsulated in Ph sensitive hydrogels, the hydrogel protects the insulin from the acidic environment of the stomach and then releases the insulin in the less acidic environment of the intestine making it available for absorption into the blood stream. The combination of making the bio-sign data conveniently and discreetly available and a discreet insulin delivery technique enables significantly enhanced compliance with measures to appropriately control glucose concentration levels and thereby reduces the adverse health consequences of poorly controlled glucose levels.

In the event that the monitor detects no anticipated changes for a prolonged period of time, indicating that the diabetic is not responding to the bio-sign data that has be transferred by the monitor or in the event that particularly alarming bio-sign levels are monitored, then the monitor expands the diversity of its communication in an attempt to ensure that the bio-sign data is available to appropriate individuals or entities other than the diabetic.

Such appropriate individuals or entities are, for purposes of this invention, referred to as care-givers and include, but are not limited to, parents, guardians, teachers, those in locus parentis, doctors, nurses and in extreme situations emergency services. Ideally such appropriate individuals or entities would have knowledge of the medical condition of the individual being monitored, including appropriate counter measures.

In such circumstances of expanded communication the data transferred by the monitor would include; a clear identification of the diabetic individual; the relevant bio-sign level or levels; appropriate counter measures; and the location of the monitor and hence of the diabetic.

Expanded communication can be achieved by the monitor by means of a wireless link to the Internet, or to a device connected to the Internet. This means is facilitated by the growing availability of wireless links, such as that commonly referred to as WiFi. Expanded communication can also be achieved by the monitor by means of text messaging. Such text messaging can be routed directly to the end recipient or by way of an intermediate device that automatically translates the text information to a compelling audio or audio/video sequence.

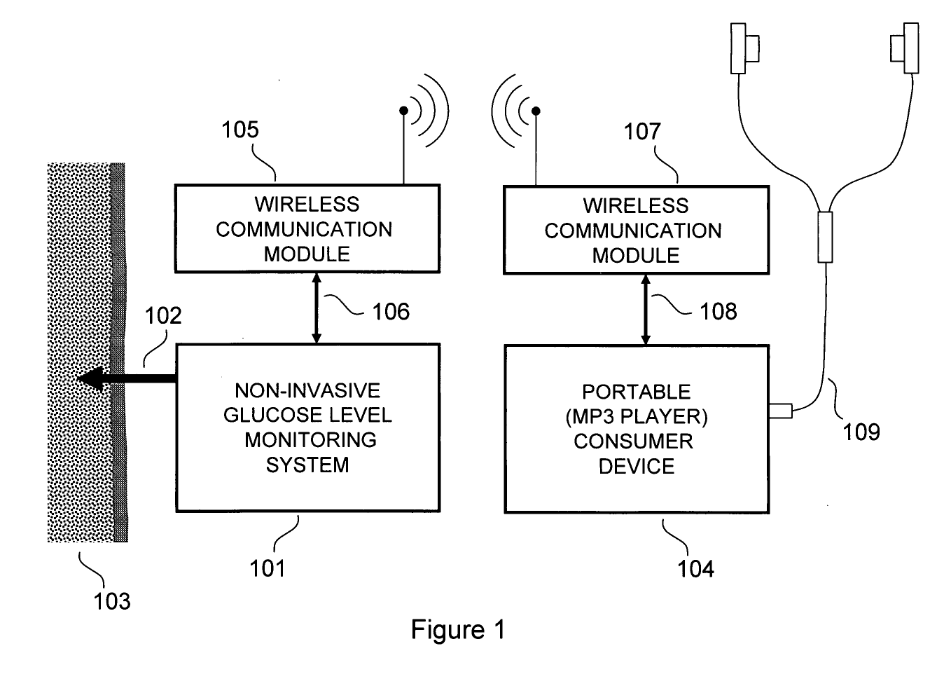

A preferred embodiment of this invention for enabling enhanced compliance with a bio-sign monitor is illustrated in and described with respect to FIG. 1. A non-invasive glucose level monitoring system 101, in general referred to as a bio-sign monitor, measures the glucose level or concentration on a continuous or very frequent basis. In the preferred embodiment, the monitoring system, or monitor 101, uses an optical probe beam 102 to analyze sub-cutaneous tissue 103 to determine the glucose level, as described in patent applications incorporated herein by reference.

Information related to the measured level is transferred to at least one consumer device, such as a portable MP3 player or iPod like consumer device. In the preferred embodiment transferring data from the monitor to the consumer device 104 (or devices) is accomplished by means of a wireless (or RF) module 105 which has a conventional bidirectional wired link 106 to the monitor 101. The wireless module 105 transfers the data to a corresponding wireless module 107 which has a conventional bidirectional wired link 108 to the consumer device 104.

The data thus transferred from the monitor, in general referred to as bio-sign data, is available to the consumer device 104. By incorporating the bio-sign data as information in a conventional information format the data can be made available to the user of the consumer device by transmitting the information by means of a conventional communication channel.

In the preferred embodiment, the bio-sign data is incorporated into an audio format and the audio is transmitted to the user of the consumer device as a conventional audio channel by means of conventional ear-phones 109 or a conventional headset. In this manner bio-sign data related to such bio-signs as glucose concentration levels can be made available to a patient, such as a diabetic, conveniently and discreetly to enable significantly enhanced compliance with measures to appropriately control glucose concentration levels.

In the preferred embodiment, the bio-sign data is initially inserted as audio information in a benign manner, such as at low volume and at suitable breaks in the audio being listened to, such as between tracks or songs, extending the duration of the break if necessary. If the monitor does not detect the anticipated changes (which should be an improvement in the bio-sign) in a timely manner. The audio message could be made more loud and made to impact the MP3 player or iPod experience thereby enhancing the likelihood of compliance.

There is an incentive to implement the counter measures indicated by the bio-sign data to reduce the impact of delivery of the bio-sign related information. Optional parental control techniques could be employed to add tamper resistance to insertion of the audio message and its escalating impact on enjoying the MP3 player or iPod experience. This provides a method for diabetics to discreetly get access to glucose concentration data and guidance in appropriate counter measures, such as administering a particular insulin or glucose dose. It also provides a mechanism for ensuring that inaction by the diabetic has a prompt undesirable penalty.

Bio-sign data can include, but is not limited to: the actual measured glucose level concentration; glucose level change trend; other measurements such as heart rate, caloric expenditure rate; recommended glucose or insulin dose in various delivery modes; recommended glucose or insulin dose modified by historical and projected behavior; a choice of glucose or insulin doses related to the diabetics anticipated future behavior; time and location of monitor when bio-sign data was acquired; proposed counter measures.

Proposed counter measures would normally be a recommended glucose or insulin dose, but could also include a recommendation to desist from a behavior such as exercise or to seek medical attention from a doctor or qualified medical personnel. In general the bio-sign data and proposed counter measures enable the diabetic to comply with controlling the glucose level.

In the event that the monitor does not detect anticipated changes for a prolonged period of time, indicating that the diabetic is not responding to the bio-sign data that has be transferred by the monitor or in the event that particularly alarming bio-sign levels are monitored, then the monitor expands the diversity of its communication in an attempt to ensure that the bio-sign data is available to appropriate individuals or entities other than the diabetic. For purposes of this invention, “capable entity” includes any individual or entity, such as a care-giver or emergency service that is capable of enabling compliance with the measures indicated by the monitor.

In the preferred embodiment, if anticipated changes are not detected, the monitor expands its communication to other devices likely to be in proximity to the monitor in an endeavor to either directly or indirectly contact informed care-givers or in extreme situations emergency services. Such other devices include other wireless enabled computers, ideally that are Internet connected, or cell phones that the RF module can communicate with.

In alternative embodiments the wireless module associated with the monitor is WiFi enabled or has the capability of communicating similar to a conventional cell phone by text messaging mode or voice mode or both. This significantly expands the capability of the monitor to enhance compliance by making the connectivity of the cellular phone system and/or the Internet available to ensure access to informed care-givers or medical emergency services.

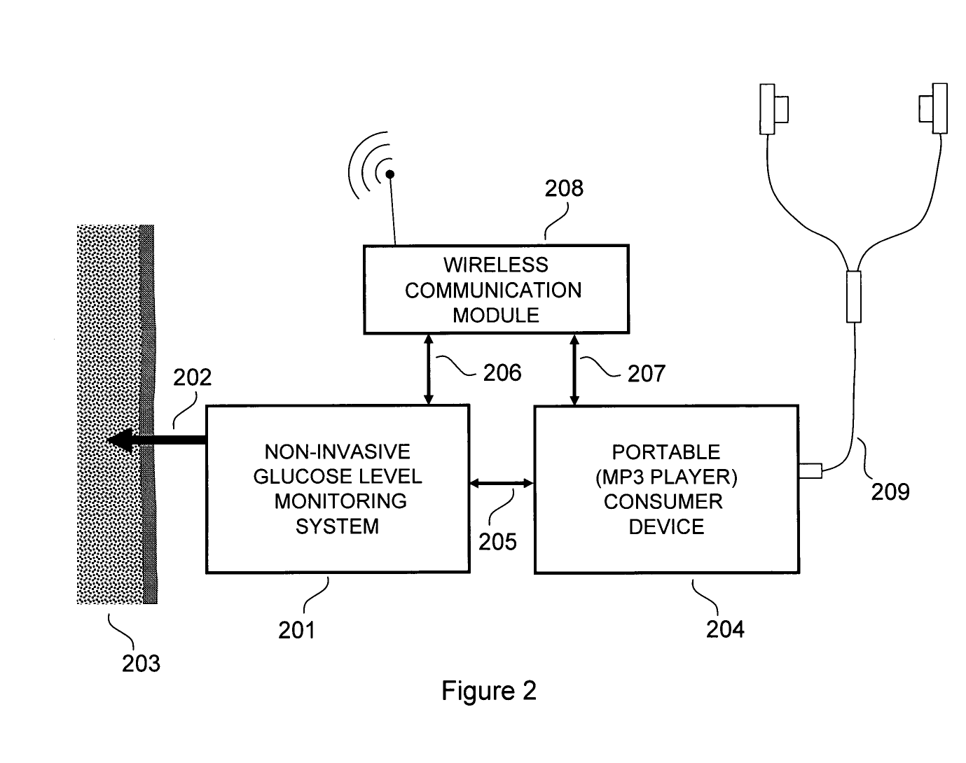

One alternative embodiment is illustrated in and described with respect to FIG. 2. As in the preferred embodiment, a non-invasive glucose level monitoring system 201 measures the glucose level or concentration on a continuous or very frequent basis using an optical probe beam 202 to analyze sub-cutaneous tissue 203 to determine the glucose level, as described in patent applications incorporated herein by reference.

The monitor is integrated with a consumer device 204, such as a cell phone, an MP3 player or iPod like device or various combinations of these types of devices. Being integrated enables transferring data over a conventional wired connection 205 between the monitor and one or more integrated devices. In this embodiment, both the monitor and the consumer device are connected to the wireless communication module 208 by means of conventional wired connections indicated by 206 and 207 or other variations of wired connections.

In this embodiment, the wireless module 208 can have multiple wireless communication modes including, but not limited to; relatively short range wireless links, such as Bluetooth; a cellular data wireless link, such as the text messaging or instant messaging cell phone mode; or fully enabled voice and data modes of conventional cell phones. In this embodiment the monitor could be integrated with a cell phone, which may or may not have an integrated iPod like device.

As in the preferred embodiment, the bio-sign data is incorporated into an audio format and the audio is transmitted to the user of the consumer device as a conventional audio channel by means of conventional ear-phones 209 or a conventional headset. In this manner bio-sign data related to such bio-signs as glucose concentration levels can be made available to a patient, such as a diabetic, conveniently and discreetly to enable significantly enhanced compliance with measures to appropriately control glucose concentration levels.

The cell phone aspect of this embodiment ensures the ability to be able to extend the diversity of care-givers to whom the monitor can communicate critical bio-sign data or related information. It also ensures the ability to connect to the Internet.

The monitor may also be connected to the Internet intermittently, for example, while the monitor battery is being recharged it could automatically be Internet connected by recharging by means of a USB port or a conventional power/data cradle. The capability of being Internet connected also enables accumulated bio-sign data to be distributed to informed care-givers such as parents or doctors. It also enables up-dated software or data to be down-loaded to the monitor.

These additional monitor capabilities enable enhanced compliance with the measures indicated by the monitor by making relevant data available in a convenient and discreet manner to the diabetic and if necessary making relevant data available to other informed care-givers.

It is understood that the above description is intended to be illustrative and not restrictive. Many of the features have functional equivalents that are intended to be included in the invention as being taught. While the preferred embodiment is described with respect to a glucose concentration level monitor and a MP3 player or iPod like consumer device the invention is not restricted to these embodiments.

Monitors could also deal with other bio-signs including, but not limited to, heart rate, blood pressure, core temperature, lactic acid levels or other metabolite levels. Other consumer devices that the monitor could transfer data to include, but are not limited to, cell phones, PDAs, portable radios, portable disc or tape players, DVD players, PCs, lap-top computers, beepers, hearing aids, spectacles with visual display units, virtual reality displays, cameras, video disc recorders, cam-corders, etc.

Such consumer devices could also have a proprietary communication channel or proprietary modifications, either hardware or software, to conventional communication channels to facilitate transferring data between the monitor and the consumer device. This invention is also not specific to monitoring bio-signs, but could enable enhanced compliance with the counter measures indicated by monitors in general, for example industrial applications in noisy environments.

Many variations and combinations of the above embodiments are possible, for example, in the embodiment of FIG. 2 where the monitor is integrated with a consumer device, such as an MP3 player or iPod allowing the bio-sign data to be transferred to the consumer device by a conventional wired connection. In this embodiment the monitor may be wired to the wireless module by means of its wired connection to the consumer device (or visa versa).

The bio-sign data need not be incorporated into a communication channel as audio, but could be a visual information in the case of a computer, DVD player, a camera or cam-corder. It could be a combination of audio and visual information. A tactile communication channel could also be used, such as the vibration mode of some cell phones.

Many of the features have functional equivalents that are intended to be included in the invention as taught. For example, monitors could communicate with consumer devices by means of fiber optics or an infra-red communication channel. Bio-sign data could be incorporated into an audio format in the digital electronic domain or in the audio domain. Other examples will be apparent to persons skilled in the art. The scope of this invention should be determined with reference to the specification, the drawings, the appended claims, along with the full scope of equivalents as applied thereto.

What is claimed is:

1. A method of enabling compliance with a bio-sign monitor, comprising:
transferring data from said monitor to at least one consumer device;
incorporating said data as information in a conventional information format;
transmitting said information by means of a conventional communication channel; and
making said information available to a capable entity to enable compliance with said bio-sign monitor.

2. The method of claim 1, wherein said monitor is a glucose concentration level monitor.

3. The method of claim 1, wherein said data is related to glucose concentration level.

4. The method of claim 1, wherein said consumer device is an iPod.

5. The method of claim 1, wherein said consumer device is a cell phone.

6. The method of claim 1, wherein said consumer device is a computer.

7. The method of claim 1, wherein said consumer device is an MP3 player.

8. The method of claim 1, wherein said conventional information format is audio.

9. The method of claim 1, wherein said conventional information format is text.

10. The method of claim 1, wherein said conventional information format is video.

11. The method of claim 1, wherein said conventional communication channel is an audio channel.

12. The method of claim 1, wherein said conventional communication channel is a wireless channel.

13. The method of claim 1, wherein said conventional communication channel is an Internet channel.

14. The method of claim 1, wherein said capable entity is the diabetic associated with said bio-sign monitor.

15. The method of claim 1, wherein said capable entity is an informed care giver.

16. The method of claim 1, wherein said capable entity is a medical emergency service.

17. A system for enabling compliance with a bio-sign monitor, said system comprising:
a module operable to transfer data from said monitor to at least one consumer device;
a module operable to incorporate said data as information in a conventional information format; and
a module operable to transmit said information by means of a conventional communication channel.

18. An apparatus for enabling compliance with a bio-sign monitor, said system comprising:
means for transferring data from said monitor to at least one consumer device;
means for incorporating said data as information in a conventional information format;
means for transmitting said information by means of a conventional communication channel; and
means for making said information available to a capable entity to enable compliance with said bio-sign monitor.

19. The apparatus of claim 18, wherein said monitor is a glucose concentration level monitor.

20. The apparatus of claim 18, wherein said data is related to glucose concentration level.

21. The apparatus of claim 18, wherein said consumer device is an iPod.

22. The apparatus of claim 18, wherein said consumer device is a cell phone.

23. The apparatus of claim 18, wherein said consumer device is a computer.

24. The apparatus of claim 18, wherein said consumer device is an MP3 player.

25. The apparatus of claim 18, wherein said conventional information format is audio.

26. The apparatus of claim 18, wherein said conventional information format is text.

27. The apparatus of claim 18, wherein said conventional information format is video.

28. The apparatus of claim 18, wherein said conventional communication channel is an audio channel.

29. The apparatus of claim 18, wherein said conventional communication channel is a wireless channel.

30. The apparatus of claim 18, wherein said conventional communication channel is an Internet channel.

31. The apparatus of claim 18, wherein said capable entity is the diabetic associated with said bio-sign monitor.

32. The apparatus of claim 18, wherein said capable entity is an informed care giver.

33. The apparatus of claim 18, wherein said capable entity is a medical emergency service.
